(19) United States
(12) Patent Application Publication
(10) Pub. No.: US 2020/0046219 A1
(43) Pub. Date: Feb. 13, 2020

(54) DEVICE ATTACHED TO A SMARTPHONE FOR OBJECTIVE VISION SCREENING

(71) Applicants: Guang-Ji Wang, Boston, MA (US); John Y. Wang, Boston, MA (US)

(72) Inventors: Guang-Ji Wang, Boston, MA (US); John Y. Wang, Boston, MA (US)

(21) Appl. No.: 16/524,292

(22) Filed: Jul. 29, 2019

Related U.S. Application Data
(60) Provisional application No. 62/716,179, filed on Aug. 8, 2018.

(51) Int. Cl. A61B 3/032; G06F 1/16; A61B 3/00; G02B 27/01

(52) U.S. Cl. CPC A61B 3/032; G02B 27/01; A61B 3/0075; G06F 1/1607

(57) ABSTRACT

A new device attached to a Smartphone has been invented to provide objective vision screening of young children including infants and newborns. The inventive device includes a main plane body which haves a cylindrical container for holding the optical elements and a house for holding the beam splitter. The main plane body is attached to a Smartphone. While the optical elements making the streak of light beam and moving it across the pupils of children, the camera of a Smartphone records the relative movement of the retinal reflex of the children eyes, which gives the information of refractive status, clarity of refractive media, alignment of the both eyes and other conditions of the eyes that may cause amblyopia. The principal of this device is similar to conventional streak retinoscopy, but it examines both eyes simultaneously and has more advantages.

### REFERENCE CITED

U.S. PATENT DOCUMENTS

| | | | | |
|---|---|---|---|---|
| 10,179,081 | January | Krenik | A61B 3/032 | 351/201 |
| | 2019 | | | |
| 10,345,591 | July | Samec et al. | A61B 3/085 | |
| | 2019 | | | |
| 10,123,693 | November | Bex et al. | A61B 3/08 | 351/221 |
| | 2018 | | | |
| 9,655,517 | May | Su et al. | A61B 3/145 | 351/206 |
| | 2017 | | | |
| 9,380,938 | May | Huang et al. | A61B 3/152 | 351/204 |
| | 2016 | | | |
| 9,149,179 | October | Barnard et al. | A61B 3/0025 | 351/200 |
| | 2015 | | | |
| 8,888,288 | November | Iravani et al.. | A61B 3/0033 | 351/222 |
| | 2017 | | | |
| 8,403,480 | March | Chen et al. | A61B 3/08 | 351/200-246 |
| | 2013 | | | |
| 6,808,267 | October | O'Neil et al. | 351/246 | 351/200 |
| | 2004 | | | |

### BACKGROUND OF THE INVENTION

### 1. Field of the Invention

The present invention relates to an ophthalmic device for objective vision screening. More particularly, the invention relates to an ophthalmic device attached to a Smartphone or like such as digital camera or iPad to detect the ocular anomalies and disorders that cause amblyopia in children.

### 2. Description of the Prior Art

Most vision screening equipment and methods currently used are subjective. They require the accurate responses from young children. Sometimes it is impossible. The subjective vision screening is to detect the abnormal visual functions by testing visual acuity and/or stereopsis. If some abnormal visual functions are found, the child will be sent to an eye care professional for a comprehensive eye examination to find the causes.

Undesirably, ophthalmic devices for vision screening tend to be large, bulky, expensive and difficult to be operated by non-professional persons.

### SUMMARY OF THE INVENTION

In view of the foregoing disadvantages inherent in the known types of vision screening equipment and methods now present in the prior art, the present invention provides a new device wherein the same can be utilized for efficiently vision screening.

The general purpose of the present invention, which will be described subsequently in greater detail, is to provide a new device attached to a Smartphone for objective vision screening that has many of the advantages of the ophthalmic instruments mentioned heretofore and many novel features that result in a new device attached to a Smartphone for objective vision screening which is not anticipated, rendered obvious, suggested, or even implied by any of the prior art, either alone or in any combination thereof.

To attain this, the present invention generally comprises a main plane body which haves a cylindrical container for holding the optical elements and a house for holding the beam splitter.

A primary object of the present invention is to provide a device for objective vision screening that will overcome the shortcomings of the prior art devices.

Another object is to provide a device for completely objective vision screening, which can examine children of any ages.

Another object is to provide a device for increasing the speed and efficiency of objective vision screening.

Another object is to provide a device that will make the examination results video recorded, which can be kept in patients files and remotely sent out for consultation.

Another object is to provide a device that will make the examination results digitally video recorded, which can be analyzed by computer programs.

Another object is to provide a device that will be able to demonstrate examination results to students for teaching purpose.

Another object is to provide a device that will make examination be easily performed by non-professional persons, such as parents, school nurse and pre-school teacher.

An additional object is to provide a device that will be light-weigh, portable and cost effective.

### BRIEF DESCRIPTION OF THE DRAWINGS

Various other objects, features and attendant advantages other present invention will become fully appreciated as the same becomes better understood when considered in conjunction with the accompanying drawings, in which like reference characters designate the same or similar parts throughout the several views, and wherein:

FIG. 1 is a side perspective view of the Device attached to Smartphone for objective vision screening.

FIG. 2 is a perspective view of the optical elements of the Device attached to Smartphone for objective vision screening.

FIG. 3 is a view of the illumination beam and the retinal reflex in the pupils of children eyes.

### DESCRIPTION OF THE PREFERRED EMBODIMENT

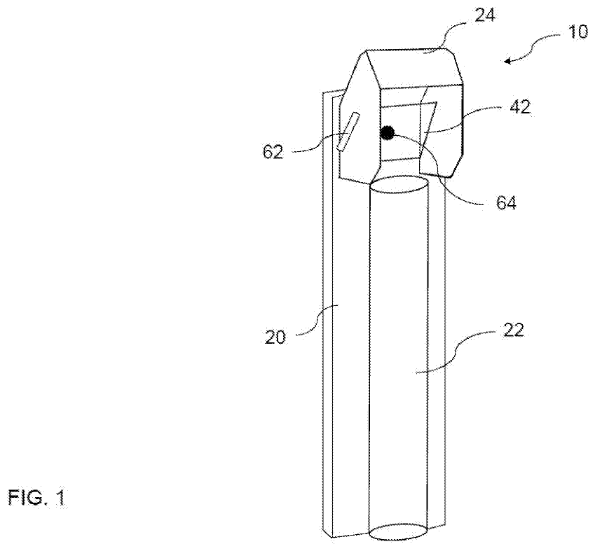

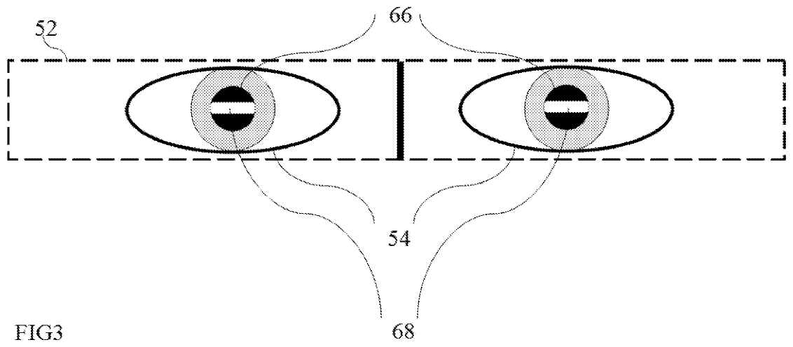

Turning now descriptively to the drawings, in which similar reference characters denote similar elements throughout the several views, FIGS. 1 through 3 illustrate the device attached to a Smartphone for objective vision screening 10, which comprises a main plane body 20 that have a cylindrical container 22 for holding the optical elements and a house 24 for holding and covering the beam splitter. In the main plane body 20 there is a peephole 64 and the nano stickers 60 on its back surface.

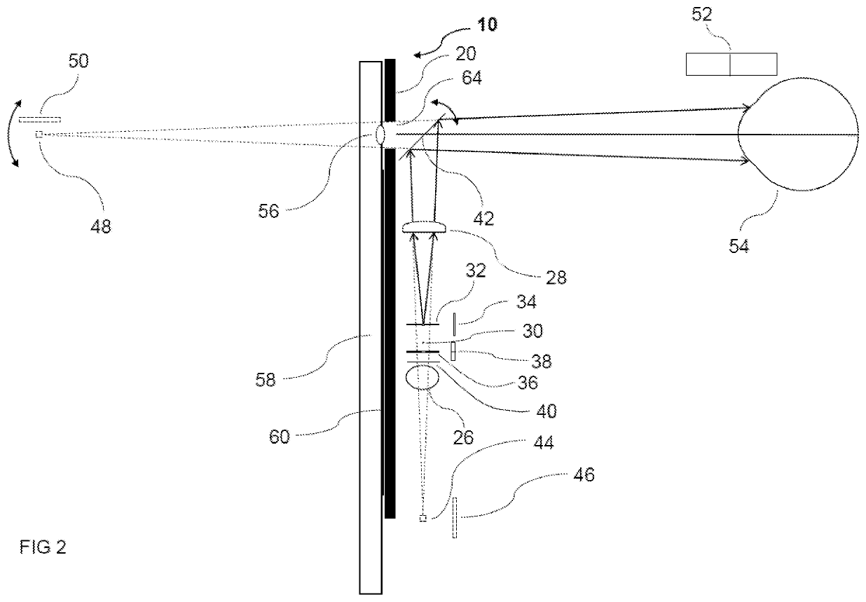

As best shown in FIG. 2 of the drawings, inside the cylindrical container 22 it holds the optical elements: a light source 26, a plus lens 28 with its primary focal point 30, a narrow slit 32 (its top view 34), a wider slit 36 with a vertical line in middle (its top view 38), and a blue-blocking filter 40. The house 24 holds a beam splitter 42 which can be rotated up and down by the handle 62. The rotation axis of the handle 62 is through the front surface of the beam splitter 42 and aligned with the center of the camera lens 56 of the Smartphone 58, and is perpendicular to the optical axis of the plus lens 28 and to the examination line, which is the line connecting the middle point of the child two eyes 54 and camera lens 56. The narrow slit 32 is located inside the primary focal point 30 of the plus lens 28, so its virtual image 44 (its top view 46) made by the plus lens 28 is further away from the plus lens 28. The beam splitter 42 turns the virtual image 44 about 90 degrees at 48 (its top view 50), and then projects towards the child's eyes 54. The wider slit 36 is located outside the primary focal point 30 of the plus lens 28, so its real image 52 made by the plus lens 28 is located in front of the plus lens 28 and turns about 90 degrees by the beam splitter 42 projecting towards the child's eyes 54. The clear, real image 52 is one meter from the camera lens 56 of the Smartphone 58.

As shown in FIG. 2, 3 of the drawing, with the beam splitter 42 rotating down and up, the beam 52 projecting on the child's eyes 54 will also move down and up. When the beam 52 enters the child's pupil 66, it will illuminate a patch of the retina, which will reflect light back through the pupils 66 and form an external image at the far point plane of the eye 54. The light seen in the child's pupil 66 is called the retinal reflex 68. With the beam splitter 42 rotating down and up, the illuminated patch of the retina also moves down and up, so that its external image will move up and down in the opposite direction.

As shown in FIG. 2, 3 of the drawing, when the external image moves across the camera lens 56, the Smartphone will capture and record the retinal reflex 68. If the external image is behind the camera lens 56, the retinal reflex 68 moves in the same direction as the beam 52 entering the child's pupil 66, and is called “with” motion. If the external image is in front of the camera lens 56, the retinal reflex 68 moves in the opposite direction from the beam 52 enter the child's pupil 66, and is called “against” motion. If the external image is at the camera lens 56, the retinal reflex 68 will not move as the beam 52 moves, and is called “no motion”.

As shown in FIG. 2 of the drawing, the device 10 is attached firmly to the Smartphone with nano stickers 60.

In use, the operator first align the peephole 64 of the device 10 with the camera lens 56 of the Smartphone 58 and press the device 10 to attach the Smartphone 58 with the nano stickers 60. Turn on the video recording function of the Smartphone 58, a round image of the peephole 64 will be seen at the center of the Smartphone screen. Adjust the camera's zoom function to 5x magnification.

Examination should be performed in a dimly lit area, not a completely dark room, to maintain large child's pupils 66. Turn on the light source 26, project the beam 52 to the forehead of the child at 1 meter away from the device 10, and place the vertical line in the middle of the beam between the child's two eyes 54. Guide the child to look at a target at about 4 meters away. A typical target can be a video cartoon to attract the child's fixation. Hold the device 10 close to the fixation line, and then rotate the handle 62 down and up a few times. During examination, the middle line image of the beam 52 should be moved along the midline between the child's two eyes 54, so that the beam 52 will equally and symmetrically enter the child's two eyes 54. When the beam 52 crosses through the pupils 66 of the child, the retinal reflex 68 in the pupils 66 of the child can be seen on the screen of the Smartphone 58 and recorded. Observe the direction of motion of the retinal reflex 68 compared with the light beam 52 motion. If it is “with” motion, it means that the eye is hyperopic, emmetropic or less than 1 D myopic; if it is “against” motion, it means that the eye is greater than 1 D myopic; if there is no motion, it means that the eye is 1 D myopic or that the eye is focusing on the device using accommodation.

If the retinal reflex 68 is too dim to detect the motion, it means the refractive error may be very high or the refractive media is clouded. Then move the device 10 close to the child so that the retinal reflex 68 becomes brighter. The brighter and wider retinal reflex 68 indicates myopia. Continue to move the device 10 closer until there is no motion, it indicates the power of myopia in diopter is equal to reciprocal of the examination distance in meters. If the retinal reflex 68 is getting brighter and narrower, it indicates high hyperopia. If the retinal reflex 68 is still dim, it may indicate cloudy refractive media, such as cataract or intraocular tumor.

The speed, width and brightness of the retinal reflex 68 will also give information of refractive status. A fast motion, with wide and bright retinal reflex 68 indicates low refractive error or emmetropia. Conversely, a slow motion with narrow and dim retinal reflex 68 indicates high refractive error.

A difference in the motion, direction, speed, width, and brightness of the retinal reflex 68 between the two eyes indicates anisometropia.

If the retinal reflex 68 has skewed motion, it indicates the eye is astigmatic with its axis not horizontal or vertical. If astigmatism is suspected in the eye, the examination should be repeated in oblique meridians (45 and 135 degrees) or in horizontal meridians. If the motion direction, brightness, or width of the retinal reflexes 68 are different between those meridians, it means that there is astigmatism in the eye.

The video recording can detect alignment of the two eyes, which can determine whether strabismus exists. The video recording can determine the clarity of the optical media of the eyes. If it is cloudy, it may indicate that the eye has cataract or tumor. The video recording can detect ptosis and anisocoria, which may interfere with vision development or indicate other medical conditions.

During the examination, a ruler or other type of small print may be put on the child's forehead to make the Smartphone camera focus better. The ruler can also be used for measurement of the interpupillary distance between the two eyes.

Putting small print or a picture in front of the device will stimulate the child to accommodate, the device may perform binocular dynamic retinoscopy, which objectively estimates the accommodative ability of the eyes.

The examination results can be judged immediately from the Smartphone screen, or can be analyzed later from the recorded files.

There is an optional test method using this device 10. Take photos when the beam 52 just touches the edge of the child's pupils 66. The photos will show the corneal reflex of the two eyes (the Hirschberg test), the comparison of the retinal reflex 68 of the two eyes, (the Bruckner test), and the shadow inside the pupils 66 (eccentric photorefraction).

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

We claim:

1. A device attached to a Smartphone for objective vision screening, comprising:
a main body having a cylindrical container for holding the optical elements and a house for the beam splitter; wherein said the optical elements are a light source, a plus lens, a narrow slit, a wider slit and a blue-blocking filter; wherein said beam splitter is a thin flat pane of glass.

2. The device attached to a Smartphone for objective vision screening of claim 1, wherein a narrow slit with a light source or a filament may be used as a narrow light source.

3. The device attached to a Smartphone for objective vision screening of claim 1, wherein the light source may have its virtue image made by a plus lens or a concave mirror.

4. The device attached to a Smartphone for objective vision screening of claim 3, wherein the virtue image of the slit or the filament may be turned about 90 degree by a beam splitter, a plane mirror or a concave mirror, and then the beam projects towards the examinee eyes.

5. The device attached to a Smartphone for objective vision screening of claim 1, wherein the beam splitter can be rotated up and down by the handle manually or by an automatic swing devices.

6. The device attached to a Smartphone for objective vision screening of claim 1, wherein the device is attached firmly to the Smartphone with nano stickers or affixed to a hard protective cell phone case so that the peephole of the device can always correctly align with the center of the camera.

7. The device attached to a Smartphone for objective vision screening of claim 1, wherein the device can be attached to a Smartphone or other digital camera or iPad.

8. The device attached to a Smartphone for objective vision screening of claim 1, wherein the device may perform dynamic retinoscopy to evaluate examinee accommodation ability.

9. The device attached to a Smartphone for objective vision screening of claim 1, wherein the device can be used to test refractive status and accommodation of animals.

10. The device attached to a Smartphone for objective vision screening of claim 1, wherein it can be used to take photos as photorefraction.
